(19) United States
(12) Patent Application Publication
(10) Pub. No.: US 2012/0328804 A1
(43) Pub. Date: Dec. 27, 2012

(54) MOLDED ARTICLES OF POLYMER-OIL COMPOSITIONS

(76) Inventors: William Maxwell Allen, Liberty Township, OH (US); Eric Bryan Bond, Maineville, OH (US); Isao Noda, Fairfield, OH (US)

(21) Appl. No.: 13/475,602

(22) Filed: May 18, 2012

Related U.S. Application Data
(60) Provisional application No. 61/488,545, filed on May 20, 2011.

(51) Int. Cl. C08L 23/12; C08L 67/00; C08L 23/06; C08K 5/103; C08K 5/101; C08K 5/1545; C08K 5/01; A61F 13/26; B32B 1/02; B65D 90/02; C08L 33/06; C08K 5/1515; B82Y 30/00

(52) U.S. Cl. 428/34.1; 524/583; 524/522; 524/599; 524/585; 524/317; 524/310; 524/300; 524/114; 524/314; 524/109; 604/11; 215/379; 977/773

(57) ABSTRACT

Molded articles formed from compositions comprising thermoplastic polymers and oils are disclosed, where the oil is dispersed throughout the thermoplastic polymer.

### CROSS REFERENCE TO RELATED APPLICATION

This application claims the benefit of U.S. Provisional Application No. 61/488,545 filed May 20, 2011.

### FIELD OF THE INVENTION

The present invention relates to molded articles formed from compositions comprising intimate admixtures of thermoplastic polymers and oils.

### BACKGROUND OF THE INVENTION

Thermoplastic polymers are used in a wide variety of applications. However, thermoplastic polymers, such as polypropylene and polyethylene pose additional challenges compared to other polymer species, with respect to formation of, for example, molded articles. For the production of molded articles, flow characteristics of the material's physical and rheological properties are important. Also, the local shear/extensional rate and shear rate are important in molded articles production and, for making molded articles with very fine structures, small defects, slight inconsistencies, or phase incompatibilities in the melt are not acceptable for a commercially viable process. Moreover, high molecular weight thermoplastic polymers cannot be easily or effectively made into molded articles with fine structures. Given their stability, it would be desirable to provide a way to easily and effectively process such high molecular weight polymers. The use of high molecular weight polymers is also beneficial for use in film and fiber applications as it generally improves strength and toughness.

Most thermoplastic polymers, such as polyethylene, polypropylene, and polyethylene terephthalate, are derived from monomers (e.g., ethylene, propylene, and terephthalic acid, respectively) that are obtained from non-renewable, fossil-based resources (e.g., petroleum, natural gas, and coal). Thus, the price and availability of these resources ultimately have a significant impact on the price of these polymers. As the worldwide price of these resources escalates, so does the price of materials made from these polymers. Furthermore, many consumers display an aversion to purchasing products that are derived from petrochemicals. In some instances, consumers are hesitant to purchase products made from non-renewable fossil-based resources. Other consumers may have adverse perceptions about products derived from petrochemicals as being “unnatural” or not environmentally friendly.

Thermoplastic polymers are often incompatible with, or have poor miscibility with additives (e.g., oils, pigments, organic dyes, perfumes, etc.) that might otherwise contribute to a reduced consumption of these polymers in the manufacture of downstream articles. Heretofore, the art has not effectively addressed how to reduce the amount of thermoplastic polymers derived from non-renewable, fossil-based resources in the manufacture of common articles employing these polymers. Accordingly, it would be desirable to address this deficiency. Existing art has combined polypropylene with additives, with polypropylene as the minor component to form cellular structures. These cellular structures are the purpose behind including renewable materials that are later removed or extracted after the structure is formed. U.S. Pat. No. 3,093,612 describes the combination of polypropylene with various fatty acids where the fatty acid is removed. The scientific paper J. Apply. Polym. Sci 82 (1) pp. 169-177 (2001) discloses use of diluents on polypropylene for thermally induced phase separation to produce an open and large cellular structure but at low polymer ratio, where the diluent is subsequently removed from the final structure. The scientific paper J. Apply. Polym. Sci 105 (4) pp. 2000-2007 (2007) produces microporous membranes via thermally induced phase separation with dibutyl phthalate and soy bean oil mixtures, with a minor component of polypropylene. The diluent is removed in the final structure. The scientific paper Journal of Membrane Science 108 (1-2) pp. 25-36 (1995) produces hollow fiber microporous membranes using soy bean oil and polypropylene mixtures, with a minor component of polypropylene and using thermally induced phase separation to produce the desired membrane structure. The diluent is removed in the final structure. In all of these cases, the diluent as described is removed to produce the final structure. These structures before the diluent is removed are oily with excessive amounts of diluent to produce very open microporous structures with pore sizes >10 μm.

A need exists for molded articles from compositions of thermoplastic polymers that allow for use of higher molecular weight and/or decreased non-renewable resource based materials, and/or incorporation of further additives, such as perfumes and dyes. A still further need is for molded articles from compositions that leave the additive present to deliver renewable materials in the final product and that can also enable the addition of further additives into the final structure, such as dyes and perfumes, for example.

### SUMMARY OF THE INVENTION

In one aspect, the invention is directed to molded articles comprising a composition comprising an intimate admixture of a thermoplastic polymer and about 5 wt % to about 40 wt % of an oil, based upon the total weight of the composition, wherein the oil has a melting point of 25° C. or less and a boiling point greater than 160° C. The molded article can be in the form of a bottle, container, tampon applicator, or applicator for insertion of a medication into a bodily orifice. The molded article can be made by a method comprising compression molding the composition. The molded article can be made by a method comprising extruding the composition. The molded article can be made by a method comprising blow molding the composition.

The thermoplastic polymer can comprise a polyolefin, a polyester, a polyamide, copolymers thereof, or combinations thereof. . The thermoplastic polymer can be selected from the group consisting of polypropylene, polyethylene, polypropylene co-polymer, polyethylene co-polymer, polyethylene terephthalate, polybutylene terephthalate, polylactic acid, polyhydroxyalkanoates, polyamide-6, polyamide-6,6, and combinations thereof. Polypropylene having a melt flow index of greater than 0.5 g/10 min or of greater than 10 g/10 min can be used. The polypropylene can have a weight average molecular weight of about 20 kDa to about 700 kDa. The thermoplastic polymer can be derived from a renewable bio-based feed stock origin, such as bio polyethylene or bio polypropylene, and/or can be recycled source, such as post consumer use. The oil can be present in the composition in an amount of about 8 wt % to about 30 wt % or about 10 wt % to about 20 wt %, based upon the total weight of the composition. The oil can comprise a lipid, which can be selected from the group consisting of a monoglyceride, diglyceride, triglyceride, fatty acid, fatty alcohol, esterified fatty acid, epoxidized lipid, maleated lipid, hydrogenated lipid, alkyd resin derived from a lipid, sucrose polyester, or combinations thereof. The oil can comprise a mineral oil, such as a linear alkane, a branched alkane, or combinations thereof. The oil can be selected from the group consisting of soy bean oil, epoxidized soy bean oil, maleated soy bean oil, corn oil, cottonseed oil, canola oil, castor oil, coconut oil, coconut seed oil, corn germ oil, fish oil, linseed oil, olive oil, oiticica oil, palm kernel oil, palm oil, palm seed oil, peanut oil, rapeseed oil, safflower oil, sperm oil, sunflower seed oil, tall oil, tung oil, whale oil, triolein, trilinolein, 1-stearo-dilinolein, 1-palmito-dilinolein, lauroleic acid, linoleic acid, linolenic acid, myristoleic acid, oleic acid, palmitoleic acid, 1,2-diacetopalmitin, and combinations thereof.

The oil can be dispersed within the thermoplastic polymer such that the oil has a droplet size of less than 10 μm, less than 5 μm, less than 1 μm, or less than 500 nm within the thermoplastic polymer. The oil can be a renewable material.

The compositions disclosed herein can further comprise an additive. The additive can be oil soluble or oil dispersible. Examples of additives include perfume, dye, pigment, surfactant, nucleating agent, clarifying agent, anti-microbial agent, nanoparticle, antistatic agent, filler, or combination thereof.

In another aspect, provided is a method of making a composition as disclosed herein, the method comprising a) mixing the thermoplastic polymer, in a molten state, with the oil, also in the molten state, to form the admixture; and b) cooling the admixture to a temperature at or less than the solidification temperature of the thermoplastic polymer in 10 seconds or less to form the composition. The method of making a composition can comprise a) melting a thermoplastic polymer to form a molten thermoplastic polymer; b) mixing the molten thermoplastic polymer and oil to form an admixture; and c) cooling the admixture to a temperature at or less than the solidification temperature of the thermoplastic polymer in 10 seconds or less. The mixing can be at a shear rate of greater than 10 s⁻¹, or about 30 to about 100 s⁻¹. The admixture can be cooled in 10 seconds or less to a temperature of 50° C. or less. The composition can be pelletized. The pelletizing can occur after cooling the admixture or before or simultaneous to cooling the admixture. The composition can be made using an extruder, such as a single- or twin-screw extruder. Alternatively, the method of making a composition can comprise a) melting a thermoplastic polymer to form a molten thermoplastic polymer; b) mixing the molten thermoplastic polymer and a oil to form an admixture; and c) extruding the molten mixture to form the finished structure, for example molded articles which solidify upon cooling.

### DETAILED DESCRIPTION OF THE INVENTION

Molded articles disclosed herein are made from compositions of an intimate admixture of a thermoplastic polymer and an oil. The term “intimate admixture” refers to the physical relationship of the oil and thermoplastic polymer, wherein the oil is dispersed within the thermoplastic polymer. The droplet size of the oil within in the thermoplastic polymer is a parameter that indicates the level of dispersion of the oil within the thermoplastic polymer. The smaller the droplet size, the higher the dispersion of the oil within the thermoplastic polymer, the larger the droplet size the lower the dispersion of the oil within the thermoplastic polymer. As used herein, the term “admixture” refers to the intimate admixture of the present invention, and not an “admixture” in the more general sense of a standard mixture of materials.

The droplet size of the oil within the thermoplastic polymer is less than 10 μm, and can be less than 5 μm, less than 1 μm, or less than 500 nm Other contemplated droplet sizes of the oil dispersed within the thermoplastic polymer include less than 9.5 μm, less than 9 μm, less than 8.5 μm, less than 8 μm, less than 7.5 μm, less than 7 μm, less than 6.5 μm, less than 6 μm, less than 5.5 μm, less than 4.5 μm, less than 4 μm, less than 3.5 μm, less than 3 μm, less than 2.5 μm, less than 2 μm, less than 1.5 μm, less than 900 nm, less than 800 nm, less than 700 nm, less than 600 nm, less than 400 nm, less than 300 nm, and less than 200 nm.

The droplet size of the oil can be measured by scanning electron microscopy (SEM) indirectly by measuring a void size in the thermoplastic polymer, after removal of the oil from the composition. Removal of the oil is typically performed prior to SEM imaging due to incompatibility of the oil and the SEM imaging technique. Thus, the void measured by SEM imaging is correlated to the droplet size of the oil in the composition.

One exemplary way to achieve a suitable dispersion of the oil within the thermoplastic polymer is by admixing the thermoplastic polymer, in a molten state, and the oil. The thermoplastic polymer is melted (e.g., exposed to temperatures greater than the thermoplastic polymer's solidification temperature) to provide the molten thermoplastic polymer and mixed with the oil. The thermoplastic polymer can be melted prior to addition of the oil or can be melted in the presence of the oil.

The thermoplastic polymer and oil can be mixed, for example, at a shear rate of greater than 10 s⁻¹. Other contemplated shear rates include greater than 10, about 15 to about 1000, about 20 to about 200, or up to 500 s⁻¹. The higher the shear rate of the mixing, the greater the dispersion of the oil in the composition as disclosed herein. Thus, the dispersion can be controlled by selecting a particular shear rate during formation of the composition.

The oil and molten thermoplastic polymer can be mixed using any mechanical means capable of providing the necessary shear rate to result in a composition as disclosed herein. Non-limiting examples of mechanical means include a mixer, such as a Haake batch mixer, and an extruder (e.g., a single- or twin-screw extruder).

The mixture of molten thermoplastic polymer and oil is then rapidly (e.g., in less than 10 seconds) cooled to a temperature lower than the solidification temperature of the thermoplastic polymer. The mixture can be cooled to less than 100° C., less than 75° C., less than 50° C., less than 40° C., less than 30° C., less than 20° C., less than 15° C., less than 10° C., or to a temperature of about 0° C. to about 30° C., about 0° C. to about 20° C., or about 0° C. to about 10° C. For example, the mixture can be placed in a low temperature liquid (e.g., the liquid is at or below the temperature to which the mixture is cooled). The liquid can be water.

Thermoplastic Polymers

Thermoplastic polymers, as used in the disclosed compositions, are polymers that melt and then, upon cooling, crystallize or harden, but can be re-melted upon further heating. Suitable thermoplastic polymers used herein have a melting temperature (also referred to as solidification temperature) from about 60° C. to about 300° C., from about 80° C. to about 250° C., or from 100° C. to 215° C.

The thermoplastic polymers can be derived from renewable resources or from fossil minerals and oils. The thermoplastic polymers derived from renewable resources are bio-based, for example such as bio produced ethylene and propylene monomers used in the production polypropylene and polyethylene. These material properties are essentially identical to fossil based product equivalents, except for the presence of carbon-14 in the thermoplastic polymer. Renewable and fossil based thermoplastic polymers can be combined together in the present invention in any ratio, depending on cost and availability. Recycled thermoplastic polymers can also be used, alone or in combination with renewable and/or fossil derived thermoplastic polymers. The recycled thermoplastic polymers can be pre-conditioned to remove any unwanted contaminants prior to compounding or they can be used during the compounding and extrusion process, as well as simply left in the admixture. These contaminants can include trace amounts of other polymers, pulp, pigments, inorganic compounds, organic compounds and other additives typically found in processed polymeric compositions. The contaminants should not negatively impact the final performance properties of the admixture, for example, causing spinning breaks during a fiber spinning process.

The molecular weight of the thermoplastic polymer is sufficiently high to enable entanglement between polymer molecules and yet low enough to be melt spinnable. Addition of the oil into the composition allows for compositions containing higher molecular weight thermoplastic polymers to be spun, compared to compositions without an oil. Thus, suitable thermoplastic polymers can have weight average molecular weights of about 1000 kDa or less, about 5 kDa to about 800 kDa, about 10 kDa to about 700 kDa, or about 20 kDa to about 400 kDa. The weight average molecular weight is determined by the specific method for each polymer, but is generally measured using either gel permeation chromatography (GPC) or from solution viscosity measurements. The thermoplastic polymer weight average molecular weight should be determined before addition into the admixture.

More specifically, however, the thermoplastic polymers preferably include polyolefins such as polyethylene or copolymers thereof, including low density, high density, linear low density, or ultra low density polyethylenes such that the polyethylene density ranges between 0.90 grams per cubic centimeter to 0.97 grams per cubic centimeter, most preferred between 0.92 and 0.95 grams per cubic centimeter. The density of the polyethylene will is determined by the amount and type of branching and depends on the polymerization technology and comonomer type. Polypropylene and/or polypropylene copolymers, including atactic polypropylene; isotactic polypropylene, syndiotactic polypropylene, and combination thereof can also be used. Polypropylene copolymers, especially ethylene can be used to lower the melting temperature and improve properties. These polypropylene polymers can be produced using metallocene and Ziegler-Natta catalyst systems. These polypropylene and polyethylene compositions can be combined together to optimize end-use properties. Polybutylene is also a useful polyolefin.

Other suitable polymers include polyamides or copolymers thereof, such as Nylon 6, Nylon 11, Nylon 12, Nylon 46, Nylon 66; polyesters or copolymers thereof, such as maleic anhydride polypropylene copolymer, polyethylene terephthalate; olefin carboxylic acid copolymers such as ethylene/acrylic acid copolymer, ethylene/maleic acid copolymer, ethylene/methacrylic acid copolymer, ethylene/vinyl acetate copolymers or combinations thereof; polyacrylates, polymethacrylates, and their copolymers such as poly(methyl methacrylates).

Other nonlimiting examples of polymers include polycarbonates, polyvinyl acetates, poly(oxymethylene), styrene copolymers, polyacrylates, polymethacrylates, poly(methyl methacrylates), polystyrene/methyl methacrylate copolymers, polyetherimides, polysulfones, or combinations thereof. In some embodiments, thermoplastic polymers include polypropylene, polyethylene, polyamides, polyvinyl alcohol, ethylene acrylic acid, polyolefin carboxylic acid copolymers, polyesters, and combinations thereof.

Biodegradable thermoplastic polymers also are contemplated for use herein. Biodegradable materials are susceptible to being assimilated by microorganisms, such as molds, fungi, and bacteria when the biodegradable material is buried in the ground or otherwise contacts the microorganisms (including contact under environmental conditions conducive to the growth of the microorganisms). Suitable biodegradable polymers also include those biodegradable materials which are environmentally-degradable using aerobic or anaerobic digestion procedures, or by virtue of being exposed to environmental elements such as sunlight, rain, moisture, wind, temperature, and the like. The biodegradable thermoplastic polymers can be used individually or as a combination of biodegradable or non-biodegradable polymers. Biodegradable polymers include polyesters containing aliphatic components. Among the polyesters are ester polycondensates containing aliphatic constituents and poly(hydroxycarboxylic) acid. The ester polycondensates include diacids/diol aliphatic polyesters such as polybutylene succinate, polybutylene succinate co-adipate, aliphatic/aromatic polyesters such as terpolymers made of butylenes diol, adipic acid and terephthalic acid. The poly(hydroxycarboxylic) acids include lactic acid based homopolymers and copolymers, polyhydroxybutyrate (PHB), or other polyhydroxyalkanoate homopolymers and copolymers. Such polyhydroxyalkanoates include copolymers of PHB with higher chain length monomers, such as C₆-C₁₂, and higher, polyhydroxyalkanaotes, such as those disclosed in U.S. Pat. Nos. RE 36,548 and 5,990,271.

An example of a suitable commercially available polylactic acid is NATUREWORKS from Cargill Dow and LACEA from Mitsui Chemical. An example of a suitable commercially available diacid/diol aliphatic polyester is the polybutylene succinate/adipate copolymers sold as BIONOLLE 1000 and BIONOLLE 3000 from the Showa High Polymer Company, Ltd. (Tokyo, Japan). An example of a suitable commercially available aliphatic/aromatic copolyester is the poly(tetramethylene adipate-co-terephthalate) sold as EASTAR BIO Copolyester from Eastman Chemical or ECOFLEX from BASF.

Non-limiting examples of suitable commercially available polypropylene or polypropylene copolymers include Basell Profax PH-835 (a 35 melt flow rate Ziegler-Natta isotactic polypropylene from Lyondell-Basell), Basell Metocene MF-650W (a 500 melt flow rate metallocene isotactic polypropylene from Lyondell-Basell), Polybond 3200 (a 250 melt flow rate maleic anhydride polypropylene copolymer from Crompton), Exxon Achieve 3854 (a 25 melt flow rate metallocene isotactic polypropylene from Exxon-Mobil Chemical), Mosten NB425 (a 25 melt flow rate Ziegler-Natta isotactic polypropylene from Unipetrol), Danimer 27510 (a polyhydroxyalkanoate polypropylene from Danimer Scientific LLC), Dow Aspun 6811A (a 27 melt index polyethylene polypropylene copolymer from Dow Chemical), and Eastman 9921 (a polyester terephthalic homopolymer with a nominally 0.81 intrinsic viscosity from Eastman Chemical).

The thermoplastic polymer component can be a single polymer species as described above or a blend of two or more thermoplastic polymers as described above.

If the polymer is polypropylene, the thermoplastic polymer can have a melt flow index of greater than 5 g/10 min, as measured by ASTM D-1238, used for measuring polypropylene. Other contemplated melt flow indices include greater than 10 g/10 min, greater than 20 g/10 min, or about 5 g/10 min to about 50 g/10 min

Oils

An oil, as used in the disclosed composition, is a lipid, mineral oil, or combination thereof, having a melting point of 25° C. or less and a boiling point of greater than 160° C. The lipid can be a monoglyceride, diglyceride, triglyceride, fatty acid, fatty alcohol, esterified fatty acid, epoxidized lipid, maleated lipid, hydrogenated lipid, alkyd resin derived from a lipid, sucrose polyester, or combinations thereof. The mineral oil can be a linear alkane, a branched alkane, or combinations thereof.

Because the oil may contain a distribution of melting temperatures to generate a peak melting temperature, the oil melting temperature is defined as having a peak melting temperature 25° C. or below as defined when >50 weight percent of the oil component melts at or below 25° C. This measurement can be made using a differential scanning calorimeter (DSC), where the heat of fusion is equated to the weight percent fraction of the oil.

The oil number average molecular weight, as determined by gel permeation chromatography (GPC), should be less than 2 kDa, preferably less than 1.5 kDa, still more preferred less than 1.2 kDa.

The amount of oil is determined via gravimetric weight loss method. The solidified mixture is placed, with the narrowest specimen dimension no greater than 1 mm, into hexane (or acetone) at a ratio of 1 g or mixture per 100 g of heaxane using a refluxing flask system. First the mixture is weighed before being placed into the reflux flask, and then the heaxane and mixtures are heated to 60° C. for 20 hours. The sample is removed and air dried for 60 minutes and a final weight determined. The equation for calculating the weight percent oil is weight % oil=([initial mass-final mass]/[initial mass])×100%

Non-limiting examples of oils contemplated in the compositions disclosed herein include castor oil, coconut oil, coconut seed oil, corn germ oil, cottonseed oil, linseed oil, fish oil, olive oil, oiticica oil, palm kernel oil, palm oil, palm seed oil, peanut oil, cottonseed oil, hempseed oil, rapeseed oil, safflower oil, soybean oil, sperm oil, sunflowerseed oil, tall oil, tung oil, whale oil, and combinations thereof. Preferred oils are corn, soy bean, canola, cottonseed, and palm kernel oil. The preferred oils can be new or processed or recycled oils, such as those used at least once, for example as used in cooking. Non-limiting examples of specific triglycerides include triglycerides such as, for example, triolein, trilinolein, 1-stearo- dilinolein, and 1,2-diacetopalmitin. Coconut oil, palm oil and palm kernel oil all have melting temperatures close to or at 25° C. and are classified as oils in the present application. The oils can be from edible plant sources and inedible plant sources. Edible plant sources, for example, include soy bean and corn. Inedible sources include jatropha oil and some variants of rapeseed oil. Other contemplated oils include 1-palmito-dilinolein, lauroleic acid, linoleic acid, linolenic acid, myristoleic acid, oleic acid, palmitoleic acid, and combinations thereof.

The oil can be from a renewable material (e.g., derived from a renewable resource). As used herein, a “renewable resource” is one that is produced by a natural process at a rate comparable to its rate of consumption (e.g., within a 100 year time frame). The resource can be replenished naturally, or via agricultural techniques. Non-limiting examples of renewable resources include plants (e.g., sugar cane, beets, corn, potatoes, citrus fruit, woody plants, lignocellulosics, hemicellulosics, cellulosic waste), animals, fish, bacteria, fungi, and forestry products. These resources can be naturally occurring, hybrids, or genetically engineered organisms. Natural resources such as crude oil, coal, natural gas, and peat, which take longer than 100 years to form, are not considered renewable resources. Mineral oil is viewed as a by-product waste stream of coal, and while not renewable, it can be considered a by-product oil.

The oil, as disclosed herein, is present in the composition at a weight percent of about 5 wt % to about 40 wt %, based upon the total weight of the composition. Other contemplated wt % ranges of the oil include about 8 wt % to about 30 wt %, with a preferred range from about 10 wt % to about 30 wt %, about 10 wt % to about 20 wt %, or about 12 wt % to about 18 wt %, based upon the total weight of the composition. Specific oil wt % contemplated include about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, and about 40 wt %, based upon the total weight of the composition.

Additives

The compositions disclosed herein can further include an additive. The additive can be dispersed throughout the composition, or can be substantially in the thermoplastic polymer portion of the thermoplastic layer or substantially in the oil portion of the composition. In cases where the additive is in the oil portion of the composition, the additive can be oil soluble or oil dispersible.

Non-limiting examples of classes of additives contemplated in the compositions disclosed herein include perfumes, dyes, pigments, nanoparticles, antistatic agents, fillers, and combinations thereof. The compositions disclosed herein can contain a single additive or a mixture of additives. For example, both a perfume and a colorant (e.g., pigment and/or dye) can be present in the composition. The additive(s), when present, is/are present in a weight percent of about 0.05 wt % to about 20 wt %, or about 0.1 wt % to about 10 wt %. Specifically contemplated weight percentages include about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, about 3 wt %, about 3.1 wt %, about 3.2 wt %, about 3.3 wt %, about 3.4 wt %, about 3.5 wt %, about 3.6 wt %, about 3.7 wt %, about 3.8 wt %, about 3.9 wt %, about 4 wt %, about 4.1 wt %, about 4.2 wt %, about 4.3 wt %, about 4.4 wt %, about 4.5 wt %, about 4.6 wt %, about 4.7 wt %, about 4.8 wt %, about 4.9 wt %, about 5 wt %, about 5.1 wt %, about 5.2 wt %, about 5.3 wt %, about 5.4 wt %, about 5.5 wt %, about 5.6 wt %, about 5.7 wt %, about 5.8 wt %, about 5.9 wt %, about 6 wt %, about 6.1 wt %, about 6.2 wt %, about 6.3 wt %, about 6.4 wt %, about 6.5 wt %, about 6.6 wt %, about 6.7 wt %, about 6.8 wt %, about 6.9 wt %, about 7 wt %, about 7.1 wt %, about 7.2 wt %, about 7.3 wt %, about 7.4 wt%, about 7.5 wt %, about 7.6 wt %, about 7.7 wt %, about 7.8 wt %, about 7.9 wt %, about 8 wt %, about 8.1 wt %, about 8.2 wt %, about 8.3 wt %, about 8.4 wt %, about 8.5 wt %, about 8.6 wt %, about 8.7 wt %, about 8.8 wt %, about 8.9 wt %, about 9 wt %, about 9.1 wt %, about 9.2 wt %, about 9.3 wt %, about 9.4 wt %, about 9.5 wt %, about 9.6 wt %, about 9.7 wt %, about 9.8 wt %, about 9.9 wt %, and about 10 wt %.

As used herein the term “perfume” is used to indicate any odoriferous material that is subsequently released from the composition as disclosed herein. A wide variety of chemicals are known for perfume uses, including materials such as aldehydes, ketones, alcohols, and esters. More commonly, naturally occurring plant and animal oils and exudates including complex mixtures of various chemical components are known for use as perfumes. The perfumes herein can be relatively simple in their compositions or can include highly sophisticated complex mixtures of natural and synthetic chemical components, all chosen to provide any desired odor. Typical perfumes can include, for example, woody/earthy bases containing exotic materials, such as sandalwood, civet and patchouli oil. The perfumes can be of a light floral fragrance (e.g. rose extract, violet extract, and lilac). The perfumes can also be formulated to provide desirable fruity odors, e.g. lime, lemon, and orange. The perfumes delivered in the compositions and articles of the present invention can be selected for an aromatherapy effect, such as providing a relaxing or invigorating mood. As such, any material that exudes a pleasant or otherwise desirable odor can be used as a perfume active in the compositions and articles of the present invention.

A pigment or dye can be inorganic, organic, or a combination thereof. Specific examples of pigments and dyes contemplated include pigment Yellow (C.I. 14), pigment Red (C.I. 48:3), pigment Blue (C.I. 15:4), pigment Black (C.I. 7), and combinations thereof. Specific contemplated dyes include water soluble ink colorants like direct dyes, acid dyes, base dyes, and various solvent soluble dyes. Examples include, but are not limited to, FD&C Blue 1 (C.I. 42090:2), D&C Red 6(C.I. 15850), D&C Red 7(C.I. 15850:1), D&C Red 9(C.I. 15585:1), D&C Red 21(C.I. 45380:2), D&C Red 22(C.I. 45380:3), D&C Red 27(C.I. 45410:1), D&C Red 28(C.I. 45410:2), D&C Red 30(C.I. 73360), D&C Red 33(C.I. 17200), D&C Red 34(C.I. 15880:1), and FD&C Yellow 5(C.I. 19140:1), FD&C Yellow 6(C.I. 15985:1), FD&C Yellow 10(C.I. 47005:1), D&C Orange 5(C.I. 45370:2), and combinations thereof.

Contemplated fillers include, but are not limited to inorganic fillers such as, for example, the oxides of magnesium, aluminum, silicon, and titanium. These materials can be added as inexpensive fillers or processing aides. Other inorganic materials that can function as fillers include hydrous magnesium silicate, titanium dioxide, calcium carbonate, clay, chalk, boron nitride, limestone, diatomaceous earth, mica glass quartz, and ceramics. Additionally, inorganic salts, including alkali metal salts, alkaline earth metal salts, phosphate salts, can be used. Additionally, alkyd resins can also be added to the composition. Alkyd resins comprise a polyol, a polyacid or anhydride, and/or a fatty acid.

Additional contemplated additives include nucleating and clarifying agents for the thermoplastic polymer. Specific examples, suitable for polypropylene, for example, are benzoic acid and derivatives (e.g. sodium benzoate and lithium benzoate), as well as kaolin, talc and zinc glycerolate. Dibenzlidene sorbitol (DBS) is an example of a clarifying agent that can be used. Other nucleating agents that can be used are organocarboxylic acid salts, sodium phosphate and metal salts (for example aluminum dibenzoate) The nucleating or clarifying agents can be added in ranges from 20 parts per million (20 ppm) to 20,000 ppm, more preferred range of 200 ppm to 2000 ppm and the most preferred range from 1000 ppm to 1500 ppm. The addition of the nucleating agent can be used to improve the tensile and impact properties of the finished admixture composition.

Contemplated surfactants include anionic surfactants, amphoteric surfactants, or a combination of anionic and amphoteric surfactants, and combinations thereof, such as surfactants disclosed, for example, in U.S. Pat. Nos. 3,929,678 and 4,259,217 and in EP 414 549, WO93/08876 and WO93/08874.

Contemplated nanoparticles include metals, metal oxides, allotropes of carbon, clays, organically modified clays, sulfates, nitrides, hydroxides, oxy/hydroxides, particulate water-insoluble polymers, silicates, phosphates and carbonates. Examples include silicon dioxide, carbon black, graphite, graphene, fullerenes, expanded graphite, carbon nanotubes, talc, calcium carbonate, bentonite, montmorillonite, kaolin, zinc glycerolate, silica, aluminosilicates, boron nitride, aluminum nitride, barium sulfate, calcium sulfate, antimony oxide, feldspar, mica, nickel, copper, iron, cobalt, steel, gold, silver, platinum, aluminum, wollastonite, aluminum oxide, zirconium oxide, titanium dioxide, cerium oxide, zinc oxide, magnesium oxide, tin oxide, iron oxides (Fe₂O₃, Fe₃O₄) and mixtures thereof. Nanoparticles can increase the strength, thermal stability, and/or abrasion resistance of the compositions disclosed herein, and can give the compositions electric properties.

It is contemplated to add waxes or that some amount of wax is present in the composition. The wax may be unrelated to the lipid present or can be a saturated version of the oil. Regardless of the nature of the wax, it's level should be less than 50 weight percent in relation to the amount of oil present. Non-limiting examples of waxes contemplated in the compositions disclosed herein include beef tallow, castor wax, coconut wax, coconut seed wax, corn germ wax, cottonseed wax, fish wax, linseed wax, olive wax, oiticica wax, palm kernel wax, palm wax, palm seed wax, peanut wax, rapeseed wax, safflower wax, soybean wax, sperm wax, sunflower seed wax, tall wax, tung wax, whale wax, and combinations thereof. Non-limiting examples of specific triglycerides include triglycerides such as, for example, tristearin, tripalmitin, 1,2-dipalmitoolein, 1,3-dipalmitoolein, 1-palmito-3-stearo-2-olein, 1-palmito-2- stearo-3-olein, 2- palmito-1-stearo-3-olein, 1,2-dipalmitolinolein, 1,2-distearo-olein, 1,3-distearo-olein, trimyristin, trilaurin and combinations thereof. Non-limiting examples of specific fatty acids contemplated include capric acid, caproic acid, caprylic acid, lauric acid, myristic acid, palmitic acid, stearic acid, and mixtures thereof. Other specific waxes contemplated include hydrogenated soy bean oil, partially hydrogenated soy bean oil, partially hydrogenated palm kernel oil, and combinations thereof. Inedible waxes from Jatropha and rapeseed oil can also be used. The wax can be selected from the group consisting of a hydrogenated plant oil, a partially hydrogenated plant oil, an epoxidized plant oil, a maleated plant oil. Specific examples of such plant oils include soy bean oil, corn oil, canola oil, and palm kernel oil. The amount of wax present can range from 0 weight percent to 40 weight percent of the composition, more preferably from 5 weight percent to 20 weight percent of the composition and most preferably from 8 weight percent to 15 weight percent of the composition.

Specific examples of mineral wax include paraffin (including petrolatum), Montan wax, as well as polyolefin waxes produced from cracking processes, preferentially polyethylene derived waxes. Mineral waxes and plant derived waxes can be combined together. Plant based waxes can be differentiated by their carbon-14 content.

Contemplated anti-static agents include fabric softeners which are known to provide antistatic benefits. For example those fabric softeners that have a fatty acyl group which has an iodine value of above 20, such as N,N-di(tallowoyl-oxy-ethyl)-N,N-dimethyl ammonium methylsulfate.

Molded Articles

Compositions as disclosed herein can be formed into molded or extruded articles. A molded article is an object that is formed when injected, compressed, or blown by means of a gas into shape defined by a female mold. Molded or extruded articles may be solid objects such as, for example, toys, or hollow objects such as, for example, bottles, containers, tampon applicators, applicators for insertion of medications into bodily orifices, medical equipment for single use, surgical equipment, or the like. Molded articles and processes for preparing them are generally described, e.g., in U.S. Pat. No. 6,730,057 and U.S. Patent Publication No. 2009/0269527, each of which is incorporated by reference herein.

The composition disclosed herein is suitable for producing container articles, such as personal care products, household cleaning products, and laundry detergent products, and packaging for such articles. Personal care products include cosmetics, hair care, skin care, and oral care products, i.e., shampoo, soap, tooth paste. Accordingly, further disclosed herein is product packaging, such as containers or bottles comprising the composition described herein. A container can refer to one or more elements of a container, e.g., body, cap, nozzle, handle, or a container in its entirety, e.g., body and cap.

The products may include a container, made from the composition, and an indicia associated with the container, which educates a potential buyer about the container. Such indicia associated with the container include a label, an insert, a page in a magazine or newspaper, a sticker, a coupon, a flyer, an in-aisle or end-of-aisle display, and point-of-sale items intended to either be taken by prospective buyers or remain in an area proximate the product.

Furthermore, the molded articles can comprise other additives, such as other polymers materials (e.g., a polypropylene, a polyethylene, a ethylene vinyl acetate, a polymethylpentene any combination thereof, or the like), a filler (e.g., glass, talc, calcium carbonate, or the like), a mold release agent, a flame retardant, an electrically conductive agent, a film anti-static agent, a pigment, an antioxidant, an impact modifier, a stabilizer (e.g., a UV absorber), wetting agents, dyes, or any combination thereof. Molded article antistatic agents include cationic, anionic, and, preferably, nonionic agents. Cationic agents include ammonium, phosphonium and sulphonium cations, with alkyl group substitutions and an associated anion such as chloride, methosulphate, or nitrate. Anionic agents contemplated include alkylsulphonates. Nonionic agents include polyethylene glycols, organic stearates, organic amides, glycerol monostearate (GMS), alkyl di-ethanolamides, and ethoxylated amines.

Processes of Making the Compositions as Disclosed Herein

Melt mixing of the polymer and oil: The polymer and oil can be suitably mixed by melting the polymer in the presence of the oil. In the melt state, the polymer and oil are subjected to shear which enables a dispersion of the oil into the polymer. In the melt state, the oil and polymer are significantly more compatible with each other.

The melt mixing of the polymer and oil can be accomplished in a number of different processes, but processes with high shear are preferred to generate the preferred morphology of the composition. The processes can involve traditional thermoplastic polymer processing equipment. The general process order involves adding the polymer to the system, melting the polymer, and then adding the oil. However, the materials can be added in any order, depending on the nature of the specific mixing system.

Haake Batch Mixer: A Haake Batch mixer is a simple mixing system with low amount of shear and mixing. The unit is composed of two mixing screws contained within a heated, fixed volume chamber. The materials are added into the top of the unit as desired. The preferred order is to add the polymer, heat to 20° C. to 120° C. above the polymer's melting (or solidification) temperature into the chamber first. Once the polymer is melted, the oil can be added and mixed with the molten polymer. The mixture is then mixed in the melt with the two mixing screws for about 5 to about 15 minutes at screw RPM from about 60 to about 120. Once the composition is mixed, the front of the unit is removed and the mixed composition is removed in the molten state. By its design, this system leaves parts of the composition at elevated temperatures before crystallization starts for several minutes. This mixing process provides an intermediate quenching process, where the composition can take about 30 seconds to about 2 minutes to cool down and solidify. Mixture of polypropylene with soy bean oil in the Haake mixture showed that greater than 20wt % of oil lead to incomplete incorporation of the oil in the polypropylene mixture, indicating that higher shear rates can lead to better incorporation of oil and greater amounts of oil able to be incorporated.

Single Screw Extruder: A single screw extruder is a typical process unit used in most molten polymer extrusion. The single screw extruder typically includes a single shaft within a barrel, the shaft and barrel engineered with certain screw elements (e.g., shapes and clearances) to adjust the shearing profile. A typical RPM range for single screw extruder is about 10 to about 120. The single screw extruder design is composed of a feed section, compression section and metering section. In the feed section, using fairly high void volume flights, the polymer is heated and supplied into the compression section, where the melting is completed and the fully molten polymer is sheared. The compression section the void volume between the flights is reduced. In the metering section the polymer the polymer is subjected to its highest shearing amount using low void volume between the flights. For this work, general purpose single screw designs were used. In this unit, a continuous or steady state type of process is achieved where the composition components are introduced at desired locations, and then subjected to temperatures and shear within target zones. The process can be considered to be a steady state process as the physical nature of the interaction at each location in the single screw process is constant as a function of time. This allows for optimization of the mixing process by enabling a zone-by-zone adjustment of the temperature and shear, where the shear can be changed through the screw elements and/or barrel design or screw speed.

The mixed composition exiting the single screw extruder can then be pelletized via extrusion of the melt into a liquid cooling medium, often water, and then the polymer strand can be cut into small pieces. There are two basic types of molten polymer pelletization process used in polymer processing: strand cutting and underwater pelletization. In strand cutting the composition is rapidly quenched (generally much less than 10 seconds) in the liquid medium then cut into small pieces. In the underwater pelletization process, the molten polymer is cut into small pieces then simultaneously or immediately thereafter placed in the presence of a low temperature liquid which rapidly quenches and crystallizes the polymer. These methods are commonly known and used within the polymer processing industry.

The polymer strands that come from the extruder are rapidly placed into a water bath, most often having a temperature range of 1° C. to 50° C. (e.g., normally is about room temperature, which is 25° C.). An alternate end use for the mixed composition is further processing into the desired structure, for example fiber spinning or injection molding. The single screw extrusion process can provide for a high level of mixing and high quench rate. A single screw extruder also can be used to further process a pelletized composition into fibers and injection molded articles. For example, the fiber single screw extruder can be a 37 mm system with a standard general purpose screw profile and a 30:1 length to diameter ratio.

Twin Screw Extruder: A twin screw extruder is the typical unit used in most molten polymer extrusion, where high intensity mixing is required. The twin screw extruder includes two shafts and an outer barrel. A typical RPM range for twin screw extruder is about 10 to about 1200. The two shafts can be co-rotating or counter rotating and allow for close tolerance, high intensity mixing. In this type of unit, a continuous or steady state type of process is achieved where the composition components are introduced at desired locations along the screws, and subjected to high temperatures and shear within target zones. The process can be considered to be a steady state process as the physical nature of the interaction at each location in the single screw process is constant as a function of time. This allows for optimization of the mixing process by enabling a zone-by-zone adjustment of the temperature and shear, where the shear can be changed through the screw elements and/or barrel design.

The mixed composition at the end of the twin screw extruder can then be pelletized via extrusion of the melt into a liquid cooling medium, often water, and then the polymer strand is cut into small pieces. There are two basic types of molten polymer pelletization process, strand cutting and underwater pelletization, used in polymer processing. In strand cutting the composition is rapidly quenched (generally much less than 10s) in the liquid medium then cut into small pieces. In the underwater pelletization process, the molten polymer is cut into small pieces then simultaneously or immediately thereafter placed in the presence of a low temperature liquid which rapidly quenches and crystallizes the polymer. An alternate end use for the mixed composition is further processing into the desired structure, for example fiber spinning or injection molding.

Three different screw profiles can be employed using a Baker Perkins CT-25 25 mm corotating 40:1 length to diameter ratio system. This specific CT-25 is composed of nine zones where the temperature can be controlled, as well as the die temperature. Four liquid injection sites as also possible, located between zone 1 and 2 (location A), zone 2 and 3 (location B), zone 4 and 5 (location C). and zone 6 and 7 (location D).

The liquid injection location are not directed heated, but indirectly through the adjacent zone temperatures. Locations A, B, C and D can be used to inject the additive. Zone 6 can contain a side feeder for adding additional solids or used for venting. Zone 8 contains a vacuum for removing any residual vapor, as needed.

Two types of regions, conveyance and mixing, are used in the CT-25. In the conveyance region, the materials are heated (including through melting which is done in Zone 1 into Zone 2 if needed) and conveyed along the length of the barrel, under low to moderate shear. The mixing section contains special elements that dramatically increase shear and mixing. The length and location of the mixing sections can be changed as needed to increase and decrease shear as needed.

Two primary types of mixing elements are used for shearing and mixing. The first are kneading blocks and the second are thermal mechanical energy elements. The simple mixing screw has 10.6% of the total screw length using mixing elements composed of kneading blocks in a single set followed by a reversing element. The kneading elements are RKB 45/5/12 (right handed forward kneading block with 45° offset and five lobes at 12 mm total element length), followed by two RKB 45/5/36 (right handed forward kneading block with 45° offset and five lobes at 36 mm total element length), that is followed by two RKB 45/5/12 and reversing element 24/12 LH (left handed reversing element 24 mm pitch at 12 mm total element length).

The Simple mixing screw mixing elements are located in zone 7. The Intensive screw is composed of additional mixing sections, four in total. The first section is single set of kneading blocks is a single element of RKB45/5/36 (located in zone 2) followed by conveyance elements into zone 3 where the second mixing zone is located. In the second mixing zone, two RKB 45/5/36 elements are directly followed by four TME 22.5/12 (thermomechanical element with 22.5 teeth per revolution and total element length of 12 mm) then two conveyance elements into the third mixing area. The third mixing area, located at the end of zone 4 into zone 5, is composed of three RKB 45/5/36 and a KB45/5/12 LH (left handed forward reversing block with 45° offset and five lobes at 12 mm total element length). The material is conveyed through zone 6 into the final mixing area comprising two TME 22.5/12, seven RKB 45/5/12, followed by SE 24/12 LH. The SE 24/12 LH is a reversing element that enables the last mixing zone to be completely filled with polymer and additive, where the intensive mixing takes place. The reversing elements can control the residence time in a given mixing area and are a key contributer to the level of mixing.

The High Intensity mixing screw is composed of three mixing sections. The first mixing section is located in zone 3 and is two RKB45/5/36 followed by three TME 22.5/12 and then conveyance into the second mixing section. Prior to the second mixing section three RSE 16/16 (right handed conveyance element with 16 mm pitch and 16 mm total element length) elements are used to increase pumping into the second mixing region. The second mixing region, located in zone 5, is composed of three RKB 45/5/36 followed by a KB 45/5/12 LH and then a full reversing element SE 24/12 LH. The combination of the SE 16/16 elements in front of the mixing zone and two reversing elements greatly increases the shear and mixing. The third mixing zone is located in zone 7 and is composed of three RKB 45/5/12, followed by two TME 22.5.12 and then three more RKB45/5/12. The third mixing zone is completed with a reversing element SE 24/12 LH.

An additional screw element type is a reversing element, which can increase the filling level in that part of the screw and provide better mixing. Twin screw compounding is a mature field. One skilled in the art can consult books for proper mixing and dispersion. These types of screw extruders are well understood in the art and a general description can be found in: Twin Screw Extrusion 2E: Technology and Principles by James White from Hansen Publications. Although specific examples are given for mixing, many different combination are possible using various element configurations to achieve the needed level of mixing.

Properties of Compositions

The compositions as disclosed herein can have one or more of the following properties that provide an advantage over known thermoplastic compositions. These benefits can be present alone or in a combination.

Shear Viscosity Reduction: Addition of an oil, e.g., SBO, to a thermoplastic polymer, e.g., Basell PH-835, reduces the viscosity of the thermoplastic polymer (here, polypropylene). Viscosity reduction is a process improvement as it can allow for effectively higher polymer flow rates by having a reduced process pressure (lower shear viscosity), or can allow for an increase in polymer molecular weight, which improves the material strength. Without the presence of the oil, it may not be possible to process the polymer with a high polymer flow rate at existing process conditions in a suitable way.

Sustainable Content: Inclusion of sustainable materials into the existing polymeric system is a strongly desired property. Materials that can be replaced every year through natural growth cycles contribute to overall lower environmental impact and are desired.

Pigmentation: Adding pigments to polymers often involves using expensive inorganic compounds that are particles within the polymer matrix. These particles are often large and can interfere in the processing of the composition. Using an oil as disclosed herein, because of the fine dispersion (as measured by droplet size) and uniform distribution throughout the thermoplastic polymer allows for coloration, such as via traditional ink compounds. Soy ink is widely used in paper publication) that does not impact processability.

Fragrance: Because the oils, for example SBO, can contain perfumes much more preferentially than the base thermoplastic polymer, the present composition can be used to contain scents that are beneficial for end-use. Many scented candles are made using SBO based or paraffin based materials, so incorporation of these into the polymer for the final composition is useful.

Morphology: The benefits are delivered via the morphology produced in production of the compositions. The morphology is produced by a combination of intensive mixing and rapid crystallization. The intensive mixing comes from the compounding process used and rapid crystallization comes from the cooling process used. High intensity mixing is desired and rapid crystallization is used to preserves the fine pore size and relatively uniform pore size distribution.

Method of Making Molded Articles

The molded articles of the compositions as disclosed herein can be prepared using a variety of techniques, such as injection molding, blow molding, compression molding, or extrusion of pipes, tubes, profiles, or cables.

Injection molding of a composition as disclosed ehrein is a multi-step process by which the composition is heated until it is molten, then forced into a closed mold where it is shaped, and finally solidified by cooling. The composition is melt processed at melting temperatures less than about 180° C., more typically less than about 160° C. to minimize unwanted thermal degradation. Three common types of machines that are used in injection molding are ram, screw plasticator with injection, and reciprocating screw devices (see Encyclopedia of Polymer Science and Engineering, Vol. 8, pp. 102-138, John Wiley and Sons, New York, 1987 (“EPSE-3”).

A ram injection molding machine is composed of a cylinder, spreader, and plunger. The plunger forces the melt in the mold. A screw plasticator with a second stage injection consists of a plasticator, directional valve, a cylinder without a spreader, and a ram. After plastication by the screw, the ram forces the melt into the mold. A reciprocating screw injection machine is composed of a barrel and a screw. The screw rotates to melt and mix the material and then moves forward to force the melt into the mold.

An example of a suitable injection molding machine is the Engel Tiebarless ES 60 TL apparatus having a mold, a nozzle, and a barrel that is divided into zones wherein each zone is equipped with thermocouples and temperature-control units. The zones of the injection molding machine can be described as front, center, and rear zones whereby the pellets are introduced into the front zone under controlled temperature. The temperature of the nozzle, mold, and barrel components of the injection molding machine can vary according to the melt processing temperature of the compositions and the molds used, but will typically be in the following ranges: nozzle, 120-170° C.; front zone, 100-160° C.; center zone 100-160° C.; rear zone 60-150° C.; and mold, 5-50° C. Other typical processing conditions include an injection pressure of about 2100 kPa to about 13,790 kPa, a holding pressure of about 2800 kPa to about 11,030 kPa, a hold time of about 2 seconds to about 15 seconds, and an injection speed of from about 2 cm/sec to about 20 cm/sec. Examples of other suitable injection molding machines include Van Dorn Model 150-RS-8F, Battenfeld Model 1600, and Engel Model ES80.

Compression molding involves charging a quantity of a composition as disclosed herein in the lower half of an open die. The top and bottom halves of the die are brought together under pressure, and then molten composition conforms to the shape of the die. The mold is then cooled to harden the plastic.

Blow molding is used for producing bottles and other hollow objects (see EPSE-3). In this process, a tube of molten composition known as a parison is extruded into a closed, hollow mold. The parison is then expanded by a gas, thrusting the composition against the walls of a mold. Subsequent cooling hardens the plastic. The mold is then opened and the article removed.

Blow molding has a number of advantages over injection molding. The pressures used are much lower than injection molding. Blow molding can be typically accomplished at pressures of 25-100 psi between the plastic and the mold surface. By comparison, injection molding pressures can reach 10,000 to 20,000 psi (see EPSE-3). In cases where the composition has a have molecular weights too high for easy flow through molds, blow molding is the technique of choice. High molecular weight polymers often have better properties than low molecular weight analogs, for example high molecular weight materials have greater resistance to environmental stress cracking. (see EPSE-3). It is possible to make extremely thin walls in products with blow molding. This means less composition is used, and solidification times are shorter, resulting in lower costs through material conservation and higher throughput. Another important feature of blow molding is that since it uses only a female mold, slight changes in extrusion conditions at the parison nozzle can vary wall thickness (see EPSE-3). This is an advantage with structures whose necessary wall thicknesses cannot be predicted in advance. Evaluation of articles of several thicknesses can be undertaken, and the thinnest, thus lightest and cheapest, article that meets specifications can be used.

Extrusion is used to form extruded articles, such as pipes, tubes, rods, cables, or profile shapes. Compositions are fed into a heating chamber and moved through the chamber by a continuously revolving screw. Single screw or twin screw extruders are commonly used for plastic extrusion. The composition is plasticated and conveyed through a pipe die head. A haul-off draws the pipe through the calibration and cooling section with a calibration die, a vacuum tank calibration unit and a cooling unit. Rigid pipes are cut to length while flexible pipes are wound. Profile extrusion may be carried out in a one step process. Extrusion procedures are further described in Hensen, F., Plastic Extrusion Technology, p 43-100.

Tampon applicators are molded or extruded in a desired shape or configuration using a variety of molding or extrusion techniques to provide an applicator comprising an outer tubular member and an inner tubular member or plunger. The outer tubular member and plunger can be made by different molding or extrusion techniques. The outer member can be molded or extruded from a composition as disclosed herein and the plunger from another material.

Generally, the process of making tampon applicators involves charging a composition as disclosed herein into a compounder, and the composition is melt blended and processed to pellets. The pellets are then constructed into tampon applicators using an injection molding apparatus. The injection molding process is typically carried out under controlled temperature, time, and speed and involves melt processing the composition such that the melted composition is injected into a mold, cooled, and molded into a desired plastic object. Alternatively, the composition can be charged directly into an injection molding apparatus and the melt molded into the desired tampon applicator.

One example of a procedure of making tampon applicators involves extruding the composition at a temperature above the melting temperature of the composition to form a rod, chopping the rod into pellets, and injection molding the pellets into the desired tampon applicator form.

The compounders that are commonly used to melt blend thermoplastic compositions are generally single-screw extruders, twin-screw extruders, and kneader extruders. Examples of commercially available extruders suitable for use herein include the Black-Clawson single-screw extruders, the Werner and Pfleiderer co-rotating twin-screw extruders, the HAAKE.RTM. Polylab System counter-rotating twin screw extruders, and the Buss kneader extruders. General discussions of polymer compounding and extrusion molding are disclosed in the Encyclopedia of Polymer Science and Engineering, Vol. 6, pp. 571-631, 1986, and Vol. 11, pp. 262-285, 1988; John Wiley and Sons, New York.

The tampon applicators can be packaged in any suitable wrapper provided that the wrapper is soil proof and disposable with dry waste. Wrappers made from biodegradable materials that create minimal or no environmental concerns for their disposal are contemplated. It is also contemplated that the tampon applicators can be packaged in wrappers made from paper, nonwoven, cellulose, thermoplastic, or any other suitable material, or combinations of these materials.

Regardless of the method by which the molded article is made, the process involves an annealing cycle. The annealing cycle time is the holding time plus cooling time of the process of making the molded article. With process conditions substantially optimized for a particular mold, an annealing cycle time is a function of the composition. Process conditions substantially optimized are the temperature settings of the zones, nozzle, and mold of the molding apparatus, the shot size, the injection pressure, and the hold pressure. Annealing cycle times provided herein are at least ten seconds less than an annealing cycle time to form a molded or extruded article from a composition as disclosed herein. A dogbone tensile bar having dimensions of ½ inch length (L) (12.7 mm)×⅛ inch width (W) (3.175 mm)× 1/16 inch height (H) (1.5875 mm) made using an Engel Tiebarless ES 60 TL injection molding machine as provided herein provides a standard article as representative of a molded or extruded article for measuring annealing cycle times herein.

The holding time is the length of time that a part is held under a holding pressure after initial material injection. The result is that air bubbles and/or sink marks, preferably both, are not visually observable on the exterior surface, preferably both exterior and interior surfaces (if applicable), with the naked eye (of a person with 20--20 vision and no vision defects) from a distance of about 20 cm from the surface of the molded or extruded article. This is to ensure the accuracy and cosmetic quality of the part. Shrinkage is taken into account by the mold design. However, shrinkage of about 1.5% to 5%, from about 1.0% to 2.5%, or 1.2% to 2.0% can occur. A shorter holding time is determined by reducing the holding time until parts do not pass the visual test described supra, do not conform to the shape and texture of the mold, are not completely filled, or exhibit excessive shrinkage. The length of time prior to the time at which such events occur is then recorded as a shorter holding time.

The cooling time is the time for the part to become solidified in the mold and to be ejected readily from the mold. The mold includes at least two parts, so that the molded article is readily removed. For removal, the mold is opened at the parting line of the two parts. The finished molded part can be removed manually from the opened mold, or it can be pushed out automatically without human intervention by an ejector system as the mold is being opened. Depending on the part geometry, such ejectors may consist of pins or rings, embedded in the mold, that can be pushed forward when the mold is open. For example, the mold can contain standard dial-type or mechanical rod-type ejector pins to mechanically assist in the ejection of the molded parts. Suitable size rod-type ejector pins are ⅛″ (3.175 mm), and the like. A shorter cooling time is determined by reducing the cooling time until parts become hung up on the mold and cannot readily pop out. The length of time prior to the time at which the part becomes hung up is then recorded as a shorter cooling time.

Processing temperatures that are set low enough to avoid thermal degradation of the composition, yet high enough to allow free flow of the composition for molding are used The composition is melt processed at melting temperatures less than about 180° C. or, more typically, less than about 160° C. to minimize thermal degradation. In general, polymers can thermally degrade when exposed to temperatures above the degradation temperature after melt for a period of time. As is understood by those skilled in the art in light of the present disclosure, the particular time required to cause thermal degradation will depend upon the particular composition, the length of time above the melt temperature (Tm), and the number of degrees above the Tm. The temperatures can be as low as reasonably possible to allow free-flow of the polymer melt in order to minimize risk of thermal degradation. During extrusion, high shear in the extruder increases the temperature in the extruder higher than the set temperature. Therefore, the set temperatures may be lower than the melt temperature of the material. Low processing temperatures also help to reduce cycle time. For example, without limitation, the set temperature of the nozzle and barrel components of the injection molding machine can vary according to the melt processing temperature of the polymeric material and the type of molds used and can be from about 20° C. below the Tm to about 30° C. above the Tm, but will typically be in the following ranges: nozzle, 120-170° C.; front zone, 100-160° C.; center zone, 100-160° C. zone, 60-160° C. The set mold temperature of the injection molding machine is also dependent on the type of composition and the type of molds used. A higher mold temperature helps polymers crystallize faster and reduces the cycle time. However, if the mold temperature is too high, the parts may come out of the mold deformed. Non-limiting examples of the mold temperature include 5-60° C. or 25-50° C.

Molding injection speed is dependent on the flow rate of the compositions. The higher flow rate, the lower viscosity, the lower speed is needed for the injection molding. Injection speed can range from about 5 cm/sec to 20 cm/sec, in one embodiment, the injection speed is 10 cm/sec. If the viscosity is high, the injection speed is increased so that extruder pressure pushes the melt materials into the mold to fill the mold. The injection molding pressure is dependent on the processing temperature and shot size. Free flow is dependent upon the injection pressure reading not higher than about 14 Mpa.

### EXAMPLES

Polymers: The primary polymers used in this work are polypropylene (PP) and polyethylene (PE), but other polymers can be used (see, e.g., U.S. Pat. No. 6,783,854, which provides a comprehensive list of polymers that are possible, although not all have been tested). Specific polymers evaluated were:
Basell Profax PH-835: Produced by Lyondell-Basell as nominally a 35 melt flow rate Ziegler-Natta isotactic polypropylene.
Basell Metocene MF-650W: Produced by Lyondell-Basell as nominally a 500 melt flow rate metallocene isotactic polypropylene.
Polybond 3200: Produced by Crompton as a nominally 250 melt flow rate maleic anhydride copolymer.
Exxon Achieve 3854: Produced by Exxon-Mobil Chemical as nominally a 25 melt flow rate metallocene isotactic polypropylene.
Mosten NB425: Produced by Unipetrol as nominally a 25 melt flow rate Ziegler-Natta isotactic polypropylene.
Danimer 27510: a polyhydroxyalkanoate copolymer from Danimer Scientific LLC.
Dow Aspun 6811A: Produced by Dow Chemical as a 27 melt index polyethylene copolymer.
Eastman 9921: Produced by Eastman Chemical as a polyester terephthalic homopolymer with a nominally 0.81 intrinsic viscosity.

Oils: Specific examples used were: Soy Bean Oil (SBO); Epoxidized soy bean oil (ESBO); Corn Oil (CO); Cottonseed Oil (CSO); and Canola Oil (CNO).

Compositions were made using a Baker Perkins CT-25 Screw, with the process conditions as noted in the below table:

TABLE 1

| | | | Ratio | | | | | | | | | | | | Poly | Oil | | | |
|---|---|---|---|---|---|---|---|---|---|---|---|---|---|---|---|---|---|---|---|
| | | | Poly- | | Twin-Screw Temperature Profile (° C.) | | | | | | | | | | Temp | Temp | Screw | Screw | Torque |
| | Polymer | Oil | mer | Oil | Z1 | Z2 | Z3 | Z4 | Z5 | Z6 | Z7 | Z8 | Z9 | Die | (° C.) | (° C.) | RPM | Type | (%) |
| 1 | 835/ | SBO | 90 | 10 | 40 | 160 | 180 | 200 | 200 | 200 | 210 | 210 | 210 | 170 | 216 | 80 | 500 | Intensive | 29 |
| | 650W | | | | | | | | | | | | | | | | | | |
| 2 | PH-835 | SBO | 90 | 10 | 40 | 160 | 180 | 200 | 200 | 200 | 210 | 210 | 210 | 170 | 214 | 80 | 500 | Intensive | 81 |
| 3 | PH-835 | SBO | 80 | 20 | 40 | 160 | 180 | 200 | 200 | 200 | 210 | 210 | 210 | 170 | 214 | 80 | 500 | Intensive | 56 |
| 4 | PH-835 | SBO | 70 | 30 | 40 | 160 | 180 | 200 | 200 | 200 | 210 | 210 | 210 | 170 | 217 | 80 | 500 | Intensive | 41 |
| 5 | PH-835 | SBO | 65 | 35 | 40 | 160 | 180 | 200 | 200 | 200 | 210 | 210 | 210 | 170 | NR | 80 | 500 | Intensive | NR |
| 6 | Achieve | SBO | 90 | 10 | 40 | 160 | 180 | 200 | 200 | 200 | 210 | 210 | 210 | 170 | 220 | 80 | 500 | Intensive | 64 |
| | 3854 | | | | | | | | | | | | | | | | | | |
| 7 | Achieve | SBO | 80 | 20 | 40 | 160 | 180 | 200 | 200 | 200 | 210 | 210 | 210 | 170 | NR | 80 | 500 | Intensive | NR |
| | 3854 | | | | | | | | | | | | | | | | | | |
| 8 | Mosten | SBO | 80 | 20 | 40 | 160 | 180 | 200 | 200 | 200 | 210 | 210 | 210 | 170 | 220 | 80 | 500 | Intensive | 44 |
| | NB425 | | | | | | | | | | | | | | | | | | |
| 9 | Mosten | SBO | 70 | 30 | 40 | 160 | 180 | 200 | 200 | 200 | 210 | 210 | 210 | 170 | 213 | 80 | 500 | Intensive | 37 |
| | NB425 | | | | | | | | | | | | | | | | | | |
| 10 | Mosten | SBO | 65 | 35 | 40 | 160 | 180 | 200 | 200 | 200 | 210 | 210 | 210 | 170 | NR | 80 | 500 | Intensive | NR |
| | NB425 | | | | | | | | | | | | | | | | | | |
| 11 | 835/ | SBO | 90 | 10 | 40 | 160 | 180 | 200 | 200 | 200 | 210 | 210 | 210 | 170 | 216 | 80 | 500 | Intensive | 46 |
| | PB3200 | | | | | | | | | | | | | | | | | | |
| 12 | 835/ | SBO | 80 | 20 | 40 | 160 | 180 | 200 | 200 | 200 | 210 | 210 | 210 | 170 | NR | 80 | 500 | Intensive | NR |
| | PB3200 | | | | | | | | | | | | | | | | | | |
| 13 | PH-835 | ESBO | 90 | 10 | 40 | 160 | 180 | 200 | 200 | 200 | 210 | 210 | 210 | 170 | 213 | 80 | 500 | Intensive | 47 |
| 14 | PH-835 | ESBO | 80 | 20 | 40 | 160 | 180 | 200 | 200 | 200 | 210 | 210 | 210 | 170 | NR | 80 | 500 | Intensive | NR |
| 15 | Achieve | ESBO | 90 | 10 | 40 | 160 | 180 | 200 | 200 | 200 | 210 | 210 | 210 | 170 | 216 | 80 | 500 | Intensive | 46 |
| | 3854 | | | | | | | | | | | | | | | | | | |
| 16 | Achieve | ESBO | 80 | 20 | 40 | 160 | 180 | 200 | 200 | 200 | 210 | 210 | 210 | 170 | NR | 80 | 500 | Intensive | NR |
| | 3854 | | | | | | | | | | | | | | | | | | |
| 17 | PH-835 | CO | 90 | 10 | 40 | 160 | 180 | 200 | 200 | 200 | 210 | 210 | 210 | 170 | 197 | 80 | 400 | High | 63 |
| 18 | PH-835 | CO | 80 | 20 | 40 | 160 | 180 | 200 | 200 | 200 | 210 | 210 | 210 | 170 | 197 | 80 | 400 | High | 50 |
| 19 | PH-835 | CO | 70 | 30 | 40 | 160 | 180 | 200 | 200 | 200 | 210 | 210 | 210 | 170 | 210 | 80 | 400 | High | 39 |
| 20 | Achieve | CO | 90 | 10 | 40 | 160 | 180 | 200 | 200 | 200 | 210 | 210 | 210 | 170 | 204 | 80 | 400 | High | 63 |
| | 3854 | | | | | | | | | | | | | | | | | | |
| 21 | Achieve | CO | 80 | 20 | 40 | 160 | 180 | 200 | 200 | 200 | 210 | 210 | 210 | 170 | 200 | 80 | 400 | High | 52 |
| | 3854 | | | | | | | | | | | | | | | | | | |
| 22 | Achieve | CO | 70 | 30 | 40 | 160 | 180 | 200 | 200 | 200 | 210 | 210 | 210 | 170 | 202 | 80 | 400 | High | 40 |
| | 3854 | | | | | | | | | | | | | | | | | | |
| 23 | PH-835 | CNO | 90 | 10 | 40 | 160 | 180 | 200 | 200 | 200 | 210 | 210 | 210 | 170 | 201 | 80 | 400 | High | 60 |
| 24 | PH-835 | CNO | 80 | 20 | 40 | 160 | 180 | 200 | 200 | 200 | 210 | 210 | 210 | 170 | 201 | 80 | 400 | High | 50 |
| 25 | PH-835 | CNO | 70 | 30 | 40 | 160 | 180 | 200 | 200 | 200 | 210 | 210 | 210 | 170 | 204 | 80 | 400 | High | 39 |
| 26 | Achieve | CNO | 90 | 10 | 40 | 160 | 180 | 200 | 200 | 200 | 210 | 210 | 210 | 170 | 206 | 80 | 400 | High | 62 |
| | 3854 | | | | | | | | | | | | | | | | | | |
| 27 | Achieve | CNO | 80 | 20 | 40 | 160 | 180 | 200 | 200 | 200 | 210 | 210 | 210 | 170 | 207 | 80 | 400 | High | 51 |
| | 3854 | | | | | | | | | | | | | | | | | | |
| 28 | Achieve | CNO | 70 | 30 | 40 | 160 | 180 | 200 | 200 | 200 | 210 | 210 | 210 | 170 | 204 | 80 | 400 | High | 41 |
| | 3854 | | | | | | | | | | | | | | | | | | |
| 29 | PH-835 | CSO | 90 | 10 | 40 | 160 | 180 | 200 | 200 | 200 | 210 | 210 | 210 | 170 | 197 | 80 | 400 | High | 60 |
| 30 | PH-835 | CSO | 80 | 20 | 40 | 160 | 180 | 200 | 200 | 200 | 210 | 210 | 210 | 170 | 196 | 80 | 400 | High | 51 |
| 31 | PH-835 | CSO | 70 | 30 | 40 | 160 | 180 | 200 | 200 | 200 | 210 | 210 | 210 | 170 | 196 | 80 | 400 | High | 39 |
| 32 | Achieve | CSO | 90 | 10 | 40 | 160 | 180 | 200 | 200 | 200 | 210 | 210 | 210 | 170 | 199 | 80 | 400 | High | 62 |
| | 3854 | | | | | | | | | | | | | | | | | | |
| 33 | Achieve | CSO | 80 | 20 | 40 | 160 | 180 | 200 | 200 | 200 | 210 | 210 | 210 | 170 | 193 | 80 | 400 | High | 51 |
| | 3854 | | | | | | | | | | | | | | | | | | |
| 34 | Achieve | CSO | 70 | 30 | 40 | 160 | 180 | 200 | 200 | 200 | 210 | 210 | 210 | 170 | 194 | 80 | 400 | High | 40 |
| | 3854 | | | | | | | | | | | | | | | | | | |
| 35 | Dani- | SBO | 95 | 5 | 40 | 170 | 180 | 180 | 180 | 180 | 180 | 180 | 180 | 170 | 177 | 80 | 500 | High | 40 |
| | mer | | | | | | | | | | | | | | | | | | |
| | 27510 | | | | | | | | | | | | | | | | | | |
| 36 | Dani- | SBO | 93 | 7 | 40 | 170 | 180 | 180 | 180 | 180 | 180 | 180 | 180 | 170 | 171 | 80 | 500 | High | 32 |
| | mer | | | | | | | | | | | | | | | | | | |
| | 27510 | | | | | | | | | | | | | | | | | | |
| 37 | Dani- | SBO | 90 | 10 | 40 | 170 | 180 | 180 | 180 | 180 | 180 | 180 | 180 | 170 | 169 | 80 | 500 | High | 22 |
| | mer | | | | | | | | | | | | | | | | | | |
| | 27510 | | | | | | | | | | | | | | | | | | |
| 38 | Aspun | SBO | 90 | 10 | 40 | 160 | 180 | 190 | 190 | 190 | 190 | 190 | 190 | 170 | 176 | 80 | 500 | High | 50 |
| | 6811A | | | | | | | | | | | | | | | | | | |
| 39 | Aspun | SBO | 80 | 20 | 40 | 160 | 180 | 190 | 190 | 190 | 190 | 190 | 190 | 170 | 179 | 80 | 500 | High | 41 |
| | 6811A | | | | | | | | | | | | | | | | | | |
| 40 | Aspun | SBO | 70 | 30 | 40 | 160 | 180 | 190 | 190 | 190 | 190 | 190 | 190 | 170 | 168 | 80 | 500 | High | 28 |
| | 6811A | | | | | | | | | | | | | | | | | | |
| 41 | East- | SBO | 85 | 15 | 40 | 220 | 260 | 270 | 290 | 290 | 290 | 290 | 280 | 250 | 262 | 80 | 600 | High | 43 |
| | man | | | | | | | | | | | | | | | | | | |
| | 9921 | | | | | | | | | | | | | | | | | | |
| 42 | East- | SBO | 80 | 20 | 40 | 220 | 260 | 270 | 290 | 290 | 290 | 290 | 280 | 250 | NR | 80 | 500 | High | NR |
| | man | | | | | | | | | | | | | | | | | | |
| | 9921 | | | | | | | | | | | | | | | | | | |

For examples 5, 7, 10, 12, 16, and 42, it was noted that the SBO was surging at the end of the CT-25 extruder. Examples 5, 7, 10, 12, 16, 39, and 41 failed to properly pelletize. Example 41 produced brittle strands.

The shear viscosity influence of adding soy bean oil to Lyondell Basell Profax PH-835 at 10, 20 and 30 wt % was measured using a capillary rheometer according to ASTM D3835 at 230° C. using a 30:1 capillary. Adding 30 wt % soy bean oil to PH-835 results in a 50% reduction in shear viscosity at 1000 s⁻¹, which results in lower flow forces and process pressures.

Examples 1-42 show the polymer plus additive tested in a stable range and to the limit As used herein, stable refers to the ability of the composition to be extruded and to be pelletized. What was observed was that during the stable composition, strands from the B&P 25 mm system could be extruded, quenched in a water bath at 5° C. and cut via a pelletizer without interruption. The twin-screw extrudiate was immediately dropped into the water bath. During stable extrusion, no significant amount of oil separated from the formulation strand (>99 wt % made it through the pelletizer). The composition became unstable when it was clear that the polymer and oil were separating from each other at the end of the twin-screw and the composition strands could not be maintained. Without being bound by theory, the polymer at this point is considered fully saturated. The saturation point can change based on the oil and polymer combination, along with the process conditions. The practical utility is that the oil and polymer remain admixed and do not separate, which is a function of the mixing level and quench rate for proper dispersion of the additive. Specific Examples where the extrusion became unstable from high oil inclusion are Example 5, 7,10, 12, 16 and 42.

Molded articles can be produced from a composition of any one of Examples 1-42.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A molded article comprising a composition comprising an intimate admixture of
(a) a thermoplastic polymer; and
(b) about 5 wt % to about 40 wt % of an oil, based upon the total weight of the composition, the oil having a melting point of 25° C. or less and a boiling point greater than 160° C.

2. The molded article of claim 1, wherein the thermoplastic polymer comprises a polyolefin, a polyester, a polyamide, copolymers thereof, or combinations thereof.

3. The molded article of claim 2, wherein the thermoplastic polymer is selected from the group consisting of polypropylene, polyethylene, polypropylene co-polymer, polyethylene co-polymer, polyethylene terephthalate, polybutylene terepthalate, polylactic acid, polyhydroxyalkanoates, polyamide-6, polyamide-6,6, and combinations thereof.

4. The molded article of claim 1, wherein the thermoplastic polymer comprises polypropylene.

5. The molded article of claim 4, wherein the polypropylene has a weight average molecular weight of about 20 kDa to about 400 kDa.

6. The molded article of claim 4, wherein the polypropylene has a melt flow index of greater than 5 g/10 min

7. The molded article of claim 6, wherein the polypropylene has a melt flow index of greater than 10 g/10 min

8. The molded article of claim 1, comprising about 8 wt % to about 30 wt % of the oil, based upon the total weight of the composition.

9. The molded article of claim 8, comprising about 10 wt % to about 20 wt % of the oil, based upon the total weight of the composition

10. The molded article of any claim 1, wherein the oil comprises a lipid.

11. The molded article of claim 10, wherein the lipid comprises a monoglyceride, diglyceride, triglyceride, fatty acid, fatty alcohol, esterified fatty acid, epoxidized lipid, maleated lipid, hydrogenated lipid, alkyd resin derived from a lipid, sucrose polyester, or combinations thereof.

12. The molded article of claim 1, wherein the oil comprises a mineral oil.

13. The molded article of claim 12, wherein the mineral oil comprises a linear alkane, a branched alkane, or combinations thereof.

14. The molded article of claim 1, wherein the oil is selected from the group consisting of soy bean oil, epoxidized soy bean oil, maleated soy bean oil, corn oil, cottonseed oil, canola oil, castor oil, coconut oil, coconut seed oil, corn germ oil, linseed oil, fish oil, olive oil, oiticica oil, palm kernel oil, palm oil, palm seed oil, peanut oil, cottonseed oil, hempseed oil, rapeseed oil, safflower oil, sperm oil, sunflower seed oil, tall oil, tung oil, whale oil, triolein, trilinolein, 1-stearo- dilinolein, 1-palmito-dilinolein, lauroleic acid, linoleic acid, linolenic acid, myristoleic acid, oleic acid, palmitoleic acid, 1,2-diacetopalmitin, and combinations thereof.

15. The molded article of any claim 1, wherein the oil is dispersed within the thermoplastic polymer such that the oil has a droplet size of less than 10 μm within the thermoplastic polymer.

16. The molded article of claim 15, wherein the droplet size is less than 5 μm.

17. The molded article of claim 16, wherein the droplet size is less than 1 μm.

18. The molded article of claim 17, wherein the droplet size is less than 500 nm.

19. The molded article of claim 1, further comprising an additive.

20. The molded article of claim 19, wherein the additive is oil soluble or oil dispersible.

21. The molded article of claim 19, wherein the additive is a perfume, dye, pigment, surfactant, nanoparticle, antistatic agent, filler, nucleating agent, or combination thereof.

22. The molded article of claim 1, wherein the oil is a renewable material.

23. The molded article of claim 1, in the form of a bottle, container, tampon applicator, or applicator for insertion of a medication into a bodily orifice.

24. The molded article of claim 1 made by a method comprising compression molding the composition.

25. The molded article of claim 1 made by a method comprising extruding the composition.

26. The molded article of claim 1 made by a method comprising blow molding the composition.
